(19) United States
(12) Patent Application Publication
(10) Pub. No.: US 2026/0316977 A1
(43) Pub. Date: Oct. 15, 2026

(54) APPARATUS AND METHOD FOR INSERTING PLUNGER RODS INTO SYRINGE BARRELS

(71) Applicant: Weiler Labeling Systems, LLC, Moorestown, NJ (US)

(72) Inventors: Richard C. POWELL, JR., West Deptford, NJ (US); John Andrew SCOTT, Oreland, PA (US); Gregory Thomas VAN NESS, Jackson, NJ (US); Francis John HOLZER, Westampton, NJ (US)

(21) Appl. No.: 19/573,578

(22) Filed: Mar. 20, 2026

Related U.S. Application Data
(60) Provisional application No. 63/788,427, filed on Apr. 14, 2025.

(51) Int. Cl. B65B 7/28; A61M 5/315

(52) U.S. Cl. CPC B65B 7/2821; A61M 5/31511; A61M 2207/10

(57) ABSTRACT

A method for inserting a plunger rod into a syringe barrel containing a fluid, wherein a plunger seal is located between the fluid and an open end of the syringe barrel, where the method involves: aligning the plunger rod with the open end of the syringe barrel; moving a distal end of the plunger rod into the syringe barrel, including moving the distal end of the plunger rod a first distance into the syringe barrel according to a first speed parameter and after moving by the first distance, moving the distal end of the plunger rod further into the syringe barrel according to a second speed parameter that is different than the first speed parameter; detecting a contact condition of the distal end of the plunger rod with the plunger seal; and responsive to the detecting, rotating the plunger rod to connect the plunger rod to the plunger seal.

### TECHNICAL FIELD

This application relates generally to devices for producing filled, ready-to-use syringes and, more particularly, to a device and method for inserting plunger rods into syringe barrels containing medicinal solutions.

### BACKGROUND

Pre-filled, ready-to-use syringes are common for delivering a variety of medicinal solutions (e.g., vaccines, diabetes medications etc.). The syringe barrel is filled with the medicinal solution to a desired level, a plunger seal is inserted into the syringe barrel and a plunger rod is inserted into the syringe barrel. In practice, the position of the plunger seal within the syringe barrels varies from syringe to syringe due to tolerance issues in the production process. This variance creates the possibility that, during plunger rod insertion, the distal end of the plunger rod will contact and move the plunger seal, making the syringe unusable. In other cases, the distal end of the plunger rod may not be inserted far enough, such that the distal end of the plunger rod does not properly connect to the plunger seal.

It would be desirable to provide a device and method that is better suited for inserting plunger rods into syringe barrels to the appropriate position corresponding to the actual plunger seal position in the syringe barrel.

### SUMMARY

In one aspect, a method is provided inserting a plunger rod into a syringe barrel containing a fluid, wherein a plunger seal is located between the fluid and an open end of the syringe barrel. The method involves the steps of: aligning the plunger rod with the open end of the syringe barrel; moving a distal end of the plunger rod into the syringe barrel, wherein the moving involves (i) moving the distal end of the plunger rod a first distance into the syringe barrel according to a first speed parameter; (ii) after moving by the first distance, moving the distal end of the plunger rod further into the syringe barrel according to a second speed parameter that is different than the first speed parameter; detecting a contact condition of the distal end of the plunger rod with the plunger seal; and responsive to the detecting, rotating the plunger rod to connect the plunger rod to the plunger seal.

In another aspect, an apparatus is provided for inserting a plunger rod into a syringe barrel containing a fluid, wherein a plunger seal is located between the fluid and an open end of the syringe barrel. The apparatus includes a plunger rod grip assembly, a syringe barrel grip assembly aligned with the plunger rod grip assembly, a prime mover connected for selectively moving the plunger rod grip assembly from a plunger rod receive location and toward the syringe barrel grip assembly and a controller. The controller is configured to effect operation of the prime mover such that, when a plunger rod is held by the plunger rod grip assembly and a syringe barrel is held by the syringe barrel grip assembly: the plunger rod grip assembly is moved axially toward the syringe barrel grip assembly until a contact condition of the plunger rod with the plunger seal is detected, wherein the controller is configured such that movement of the plunger rod grip assembly axially toward the syringe barrel grip assembly involves: (i) moving the plunger rod grip assembly axially toward the syringe barrel grip assembly at a first average speed over a first distance; (ii) after moving by the first distance, moving the plunger rod grip assembly toward the syringe barrel grip assembly at a second average speed until detection of the contact condition; and responsive to detection of the contact condition, the plunger rod grip assembly is rotated to rotate the plunger rod to connect the plunger rod to the plunger seal.

In another aspect, a method of inserting a plunger rod into a syringe barrel containing a fluid, wherein a plunger seal is located between the fluid and an open end of the syringe barrel, the method comprising the steps of: aligning the plunger rod with the open end of the syringe barrel; moving a distal end of the plunger rod axially into the syringe barrel under a speed control profile that reduces an axial speed of the axial movement after a set axial distance; detecting a contact condition of the distal end of the plunger rod with the plunger seal; and responsive to the detecting, rotating the plunger rod to connect the plunger rod to the plunger seal.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1 shows a top plan view of a syringe assembly and labeling machine;

FIGS. 2 and 3 show side elevation views of the machine;

FIGS. 4-5 show perspective views of main turret regions of the machine;

FIG. 6 shows a perspective view of a plunger rod gripping region;

FIG. 7 shows a gripped plunger rod;

FIG. 8 shows a perspective view of a gripped plunger rod aligned with a syringe barrel (shown in dashed line);

FIG. 9 shows a perspective view of a gripped syringe barrel and associated actuation assembly;

FIGS. 10A-10H show a sequence of plunger rod install into a pre-filled syringe barrel; and

FIGS. 11A-11C show a sequence of syringe barrel gripping.

### DETAILED DESCRIPTION

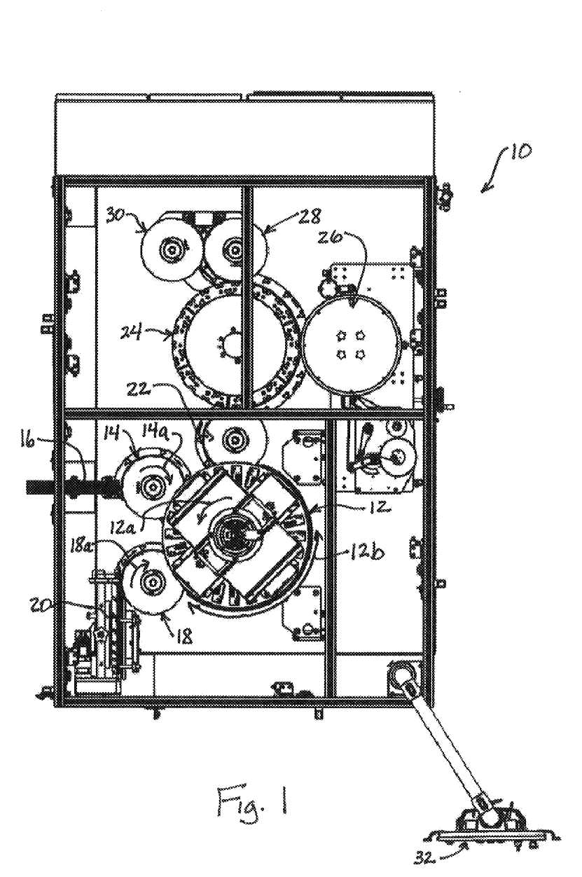

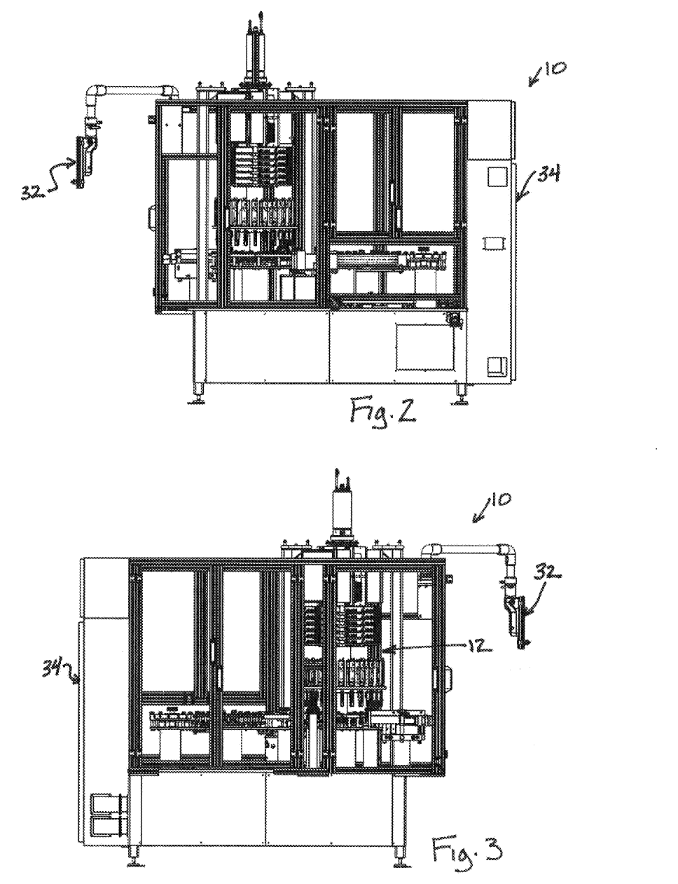

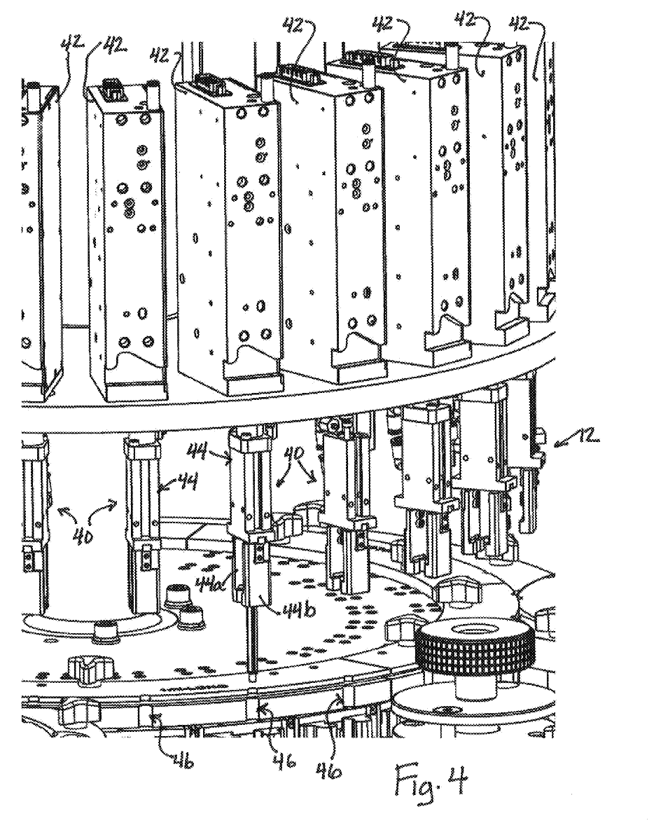

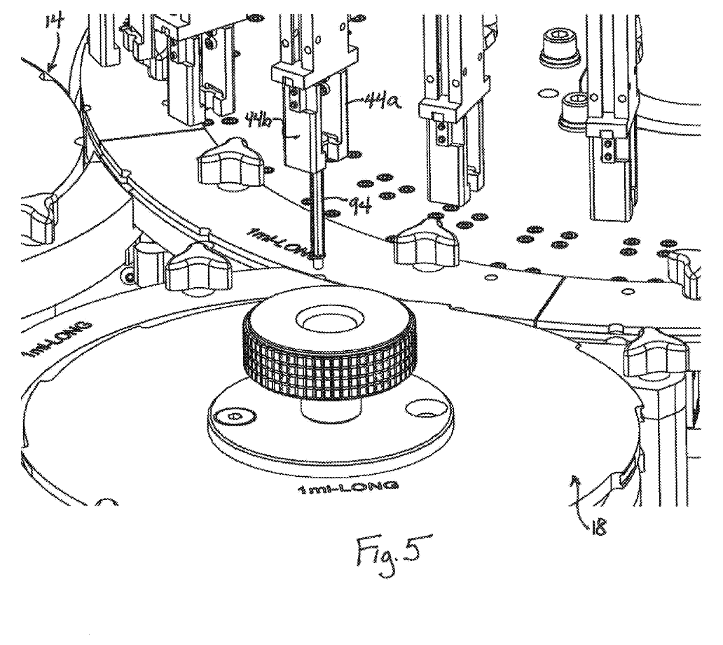

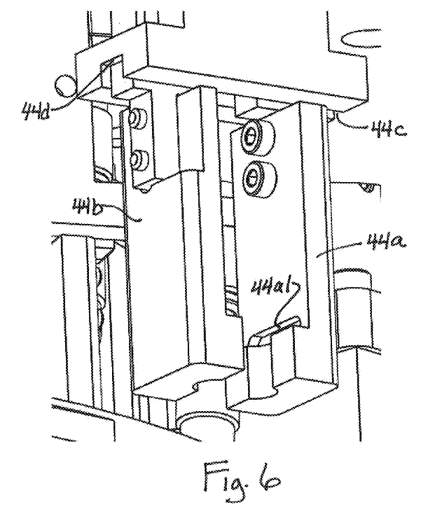

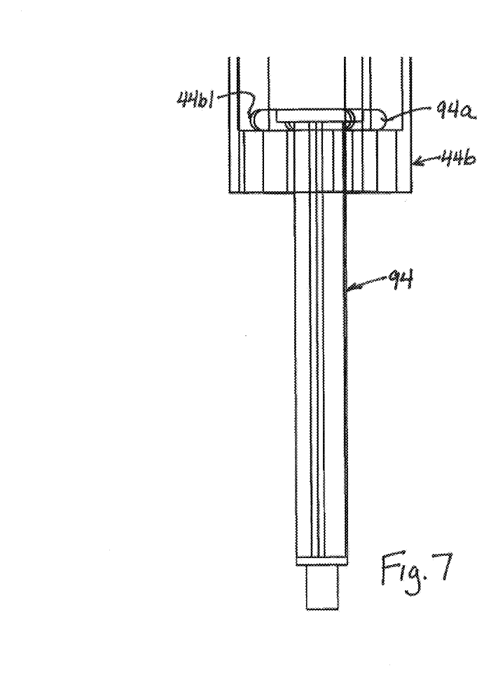

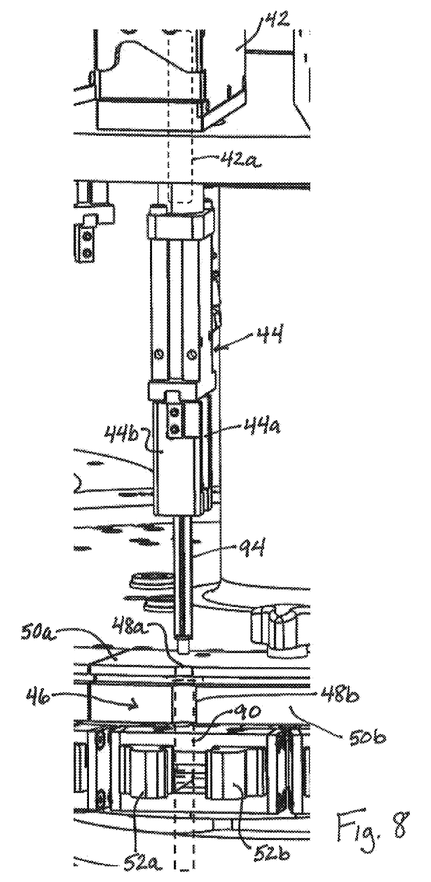

Referring to FIGS. 1-11C, a syringe assembly and labeling machine 10 includes a main turret 12 that receives both pre-filled syringe barrels 90, each having a plunger seal 92 disposed therein, and plunger rods 94 for insertion into the syringe barrels. The syringe barrels 90 may include an integrated needle that is covered by a protective cap 90b. The main turret 12 rotates in direction 12a. The plunger rods 94 transfer into the main turret 12 via infeed turret 14 rotating in direction 14a, with a plunger rod infeed chute 16 feeding the plunger rods to the infeed turret 14. The pre-filled syringe barrels transfer into the main turret 12 via infeed turret 18 rotating in direction 18a, with a syringe infeed screw 20 delivering the pre-filled syringe barrels to the infeed turret 18. The plunger rod and pre-filled syringe barrel infeeds are coordinated such that each plunger rod will be axially aligned with the open end of a respective syringe barrel.

As the components are rotated through region 12b of the main turret 12, the plunger rods are inserted into the syringe barrels and into connection with the plunger seals. The assembled syringes leave the main turret via the outfeed turret 22, which in turn delivers the assembled syringes to rotary trunnion 24 that rotates the assembled syringes as labels are applied by a vacuum drum 26. The assembled syringes then exit via one of a good product transfer dial 28 or a rejected product transfer dial 30.

An HMI interface 32 for the machine is provided, and may enable selection of numerous pre-programmed operating programs (e.g., as needed for different syringe barrels and plungers) stored in the machine controller 34. The HMI and controller 34 may also be configured to allow for manual adjustment of various parameters of the machine (e.g., speed of throughput, distance and speed of plunger rod movement during insertion into the syringe barrels, etc.). As used herein, the term “controller” is intended to broadly encompass any circuit (e.g., solid state, application specific integrated circuit (ASIC), an electronic circuit, a combinational logic circuit, a field programmable gate array (FPGA)), processor(s) (e.g., shared, dedicated, or group – including hardware or software that executes code), software, firmware, PLC, PC-based controls and/or other components, or a combination of some or all of the above, that carries out the control functions of the machine or the control functions of any component thereof.

As described in more detail below, the machine 10 provides a unique and advantageous arrangement and method for inserting the plunger rods into the prefilled syringe barrels and into connection with the plunger seals.

In this regard, the main turret 12 is rotatable and carries a plurality of plunger rod apply stations 40 equally spaced apart circumferentially around the main turret 12. At each plunger rod apply station 40, a rotary linear actuator 42 is provided and includes an axially movable and rotatable shaft 42a that is connected to the top of a plunger rod grip assembly 44. The plunger rod grip assembly will move axially with the shaft 42a and will also rotate with the shaft 42a. The rotary linear actuator 42 enables selective and settable speed control for both axial movement of the shaft 42a and rotation of the shaft 42a, and monitoring of both distal and rotary movement of the shaft 42a. The rotary linear actuator 42a also incorporates feedback sensing of both linear and rotary position error of the shaft 42a. By way of example, in embodiments, an LAR31 series rotary linear actuator available from SMAC Corporation may be used. However, other rotary linear actuators could be used.

Each plunger rod grip assembly includes 44 a set of tooling arms 44a, 44b that are movable along respective slots 44c, 44d (e.g., under pneumatic control) so as to operate as plunger rod gripper arms. The gripper arms 44a, 44b are movable between plunger rod receiving locations (e.g., spaced apart by a standby distance, so as to be “open”) and plunger rod gripping locations (e.g., spaced apart by a distance lesser than the standby distance, so as to be “closed”) that will grip and hold the plunger rods. The gripper arms 44a, 44b include inwardly facing recesses 44a1, 44b1 into which edge portions of the push plate 94a of the plunger rod 94 will fit when the plunger rod is gripped for install, aiding in the gripping operation.

Each plunger rod apply station 40 also includes syringe barrel holding regions 46 spaced apart circumferentially around the main turret, with each holding region 46 in alignment with a respect plunger rod grip assembly 44. Each holding region 46 includes one or more recessed portions 48a, 48b along the perimeter of one or more rotatable plate or rail elements 50a, 50b. Barrel gripping arms 52a, 52b are located below the recessed portions and are moveable (e.g., pivotable and/or linearly slidable) between barrel receiving locations (spaced apart by a standby distance, so as to be “open”) and barrel gripping locations (spaced apart by a distance lesser than the standby distance, so as to be “closed”) that will grip and hold the syringe barrels. In one embodiment, actuation assemblies 54 are arranged to move the barrel gripping arms 52a, 52b from the barrel receiving locations to the barrel gripping locations, and vice versa, based upon interaction with a cam feature (e.g., a cam slot or perimeter surface of a stationary cam rail/plate 56 about which the assemblies 54 move with the holding regions 46).

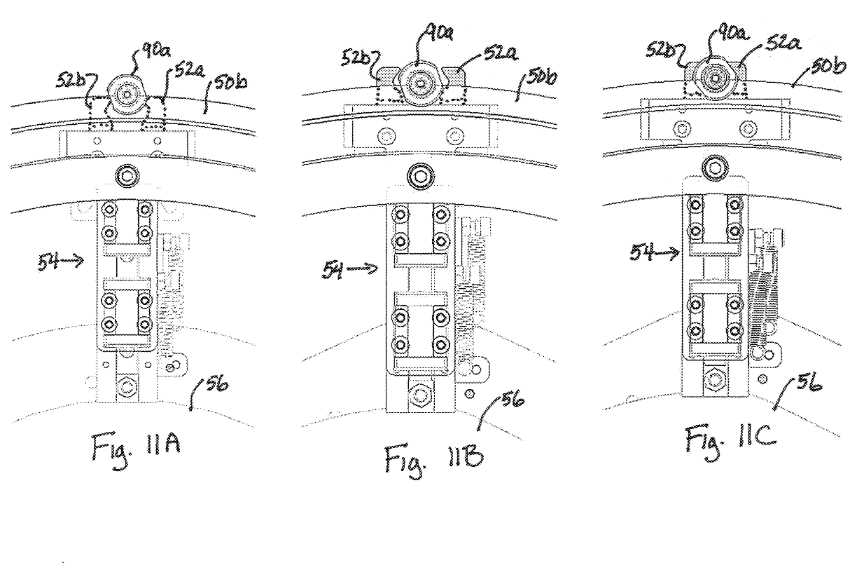

In the illustrated embodiment, the movement of the barrel gripping arms 52a, 52b is as depicted in FIGS. 11A-11C. In FIG. 11A, the barrel gripping arms 52a, 52b are in both an open and radially retracted position (e.g., retracted relative to the radially outer peripheral edge of the plate or rail element 52b). In FIG. 11B, the actuation assembly 54 effects a radially outward movement of the barrel gripping arms 52a, 52b, while the barrel gripping arms remain open, so that the barrel gripping arms 52a, 52b move along both sides of the syringe barrel. In FIG. 11C, the barrel gripping arms 52a, 52b move toward each other, into the closed position, the grip and hold the syringe barrel.

The above machine is configured (e.g., via the controller) to install plunger rods according to the following method.

A plunger rod 94 moves into a particular plunger rod applying station 40 via infeed turret 14, while the grip assembly 44 is raised and the gripper arms 44a, 44b are open. The gripper arms 44a, 44b are actuated to grip the plunger rod. As the main turret 12 continues to rotate with the gripped plunger rod, the particular plunger rod applying station comes into position to receive a pre-filled syringe barrel 90 from the infeed turret 18, with the gripping arms 52a, 52b open, and the gripping arms 52a, 52b are closed to grip the syringe barrel, while the grip rim 90a of the syringe barrel is supported on the plate element 50b. At this point, the plunger rod 94 is in axial alignment with the open end of the syringe barrel 90, such that the plunger rod 94 is ready to be inserted into the syringe barrel.

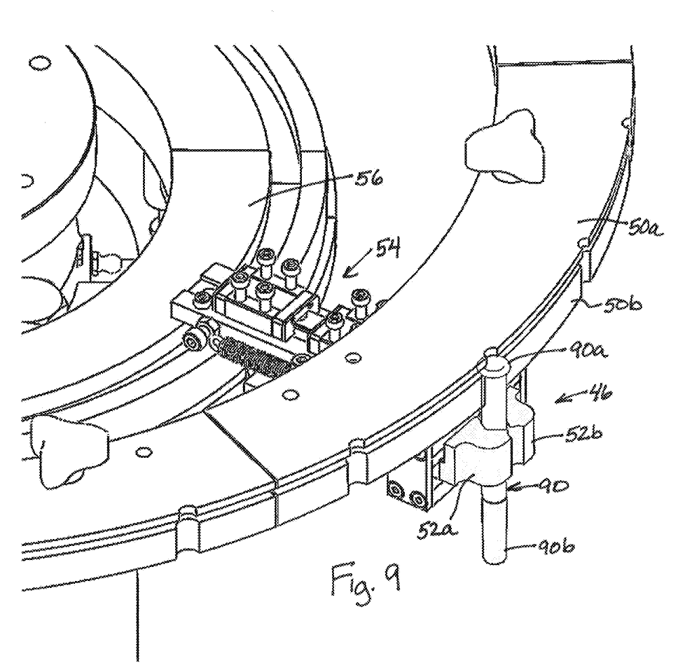

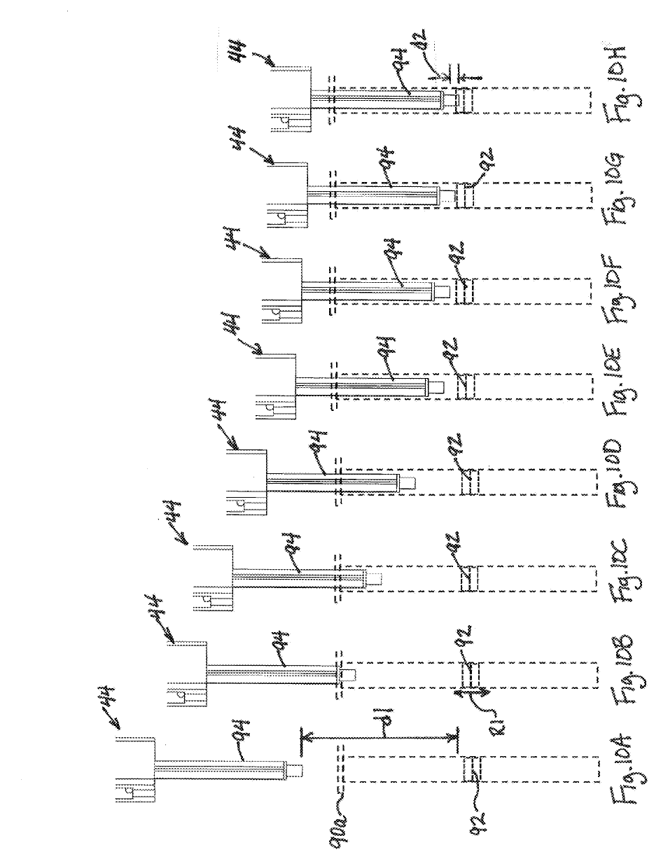

As the main turret 12 rotates the particular plunger rod applying station through region 12b, the rotary liner actuator 42 associated with the station is operated to install the plunger, as describe below with reference to FIGS. 10A-10H.

The plunger rod grip assembly 44 is moved axially toward the syringe barrel 90 to move the distal end of the plunger rod 94 into the syringe barrel 90.

A contact condition of the distal end of the plunger rod 94 with the plunger seal 92 is detected (e.g., FIG. 10H). The contact condition may be detected when the axial position error of the shaft of the rotary linear actuator exceeds a set value. The axial movement of the plunger rod grip assembly is carried out with an applied axial force that is insufficient to cause movement of the plunger seal 92 along the syringe barrel 90 when the plunger rod contacts the plunger seal.

Responsive to the detecting, the rotary linear actuator operates such that moving of the distal end of the plunger rod 94 into the syringe barrel is stopped, and the plunger rod grip assembly 44 is rotated to rotate the plunger rod 94 to connect the plunger rod 94 to an opening of the plunger seal 90.

Advantageously, the axial moving of the plunger rod into contact with the plunger seal involves a multi-step sequence according to a speed control profile that reduces an axial speed of the axial movement after a set axial distance. For example, initially, the distal end of the plunger rod 94 is moved a first distance d1 into the syringe barrel according to a first speed parameter (per FIGS. 10A-10F). As shown, the moving by the first distance d1 leaves the distal end of the plunger rod 94 short of the plunger seal 90.

After moving by the first distance d1, the rotary linear actuator moves the distal end of the plunger rod 94 further into the syringe barrel 90 according to a second speed parameter that is different than the first speed parameter. Here, the moving of the distal end of the plunger rod 94 further into syringe barrel 90 includes moving the distal end of the plunger rod 94 until detection of the contact condition (per FIGS. 10G-10H).

The first speed parameter results in a first average speed of the distal end of the plunger rod over the first distance d1, and the second speed parameter results in a second average speed over the plunger rod over a second distance d2. The first average speed is faster than the second average speed. For example, the first average speed may be at least 25% greater than the second average speed (e.g., at least 35% greater than the second average speed or at least 45% greater than the second average speed or at least 55% greater than the second average speed or at least 65% greater than the second average speed). In other embodiments, different speed control profiles could be implemented.

The rotating of the plunger rod 94 is carried out until a predetermined rotational threshold is reached, at which point the rotating is stopped, and a secondary, higher torque is applied for a set amount of time. The set torque for the initial rotation and the secondary tightening are both insufficient to rotate the plunger seal 92 relative to the syringe barrel 90. The predetermined rotational threshold may be a predetermined rotational movement distance (e.g., 540 degrees or 630 degrees or 720 degrees, or some other number of degrees).

In other contemplated embodiments, force feedback may be used to detect the contact condition of plunger rod with the plunger seal and/or to establish when to cease the rotation of the plunger rod.

At this point, the plunger rod gripper arms 44a, 44b are moved back to the open condition to release the plunger rod 94 and move clear of the plunger rod push plate 94a, and the gripping assembly can then be axially raised back into position to receive another plunger rod the next time it passes the infeed turret 14. As the assembled plunger rod and pre-filled syringe barrel continue to rotate, is exits the main turret 12 via the outfeed turret 22.

Notably, the multi-step sequence of axial moving of the distal end of plunger rod 94 into the syringe barrel at an initial “fast” speed into a position that is known will be short of the plunger seal 92 (regardless of the exact axial position of the plunger seal 42 along a range R1 of known possible positions), and the subsequent movement of the distal end at a “slow” speed until contact with the plunger seal is detected assures that: (i) the speed of install is maximized (due to use of the fast speed), (ii) the contact of the plunger rod distal end with the plunger seal actually takes place and does not cause axial movement of the plunger seal (due to use of the “slow” speed in combination with detection of the contact condition).

It is to be clearly understood that the above description is intended by way of illustration and example only, is not intended to be taken by way of limitation, and that other changes and modifications are possible.

1. A method of inserting a plunger rod into a syringe barrel containing a fluid, wherein a plunger seal is located between the fluid and an open end of the syringe barrel, the method comprising the steps of:
aligning the plunger rod with the open end of the syringe barrel;
moving a distal end of the plunger rod into the syringe barrel, wherein the moving involves:
(i) moving the distal end of the plunger rod a first distance into the syringe barrel according to a first speed parameter,
(ii) after moving by the first distance, moving the distal end of the plunger rod further into the syringe barrel according to a second speed parameter that is different than the first speed parameter;
detecting a contact condition of the distal end of the plunger rod with the plunger seal; and
responsive to the detecting, rotating the plunger rod to connect the plunger rod to the plunger seal.

2. The method of claim 1, wherein, responsive to the detecting, the moving of the distal end of the plunger rod into the syringe barrel is stopped.

3. The method of claim 1, wherein the moving by the first distance leaves the distal end of the plunger rod short of the plunger seal, wherein the moving of the distal end of the plunger rod further into syringe barrel includes moving the distal end of the plunger rod until detection of the contact condition.

4. The method of claim 3, wherein the contact condition is detected when a position error of the rotary linear actuator exceeds a set value as the distal end of the plunger rod contacts the plunger seal.

5. The method of claim 4, wherein the set force of contact is insufficient to cause movement of the plunger seal along the syringe barrel.

6. The method of claim 3, wherein the first speed parameter results in a first average speed of the distal end of the plunger rod over the first distance, wherein the second speed parameter results in a second average speed over the plunger rod over a second distance, wherein the first average speed is higher than the second average speed.

7. The method of claim 6, wherein the first average speed is at least 25% greater than the second average speed.

8. The method of claim 1, wherein the rotating continues until a predetermined rotational threshold is reached, at which point the rotating is stopped, and a secondary tightening torque is applied for a set amount of time.

9. The method of claim 8, wherein the rotating and application of the secondary tightening torque are carried out such that the plunger seal does not rotate relative to the syringe body.

10. The method of claim 1, wherein a rotary linear actuator assembly that holds the plunger rod carries out the moving, the detecting and the rotating.

11. The method of claim 10, wherein the rotary linear actuator assembly is carried by a rotating turret.

12. A method of inserting a plunger rod into a syringe barrel containing a fluid, wherein a plunger seal is located between the fluid and an open end of the syringe barrel, the method comprising the steps of:
aligning the plunger rod with the open end of the syringe barrel;
moving a distal end of the plunger rod axially into the syringe barrel under a speed control sequence that reduces an axial speed of the axial movement after a set axial distance of movement of the distal end into the syringe barrel;
detecting a contact condition of the distal end of the plunger rod with the plunger seal; and
responsive to the detecting, rotating the plunger rod to connect the plunger rod to the plunger seal.

13. An apparatus for inserting a plunger rod into a syringe barrel containing a fluid, wherein a plunger seal is located between the fluid and an open end of the syringe barrel, the apparatus comprising:
a plunger rod grip assembly;
a syringe barrel grip assembly aligned with the plunger rod grip assembly;
a prime mover connected for selectively moving the plunger rod grip assembly from a plunger rod receive location and toward the syringe barrel grip assembly;
a controller configured to effect operation of the prime mover such that, when a plunger rod is held by the plunger rod grip assembly and a syringe barrel is held by the syringe barrel grip assembly:
the plunger rod grip assembly is moved axially toward the syringe barrel grip assembly until a contact condition of the plunger rod with the plunger seal is detected, wherein the controller is configured such that movement of the plunger rod grip assembly axially toward the syringe barrel grip assembly involves:
(i) moving the plunger rod grip assembly axially toward the syringe barrel grip assembly at a first average speed over a first distance;
(ii) after moving by the first distance, moving the plunger rod grip assembly toward the syringe barrel grip assembly at a second average speed until detection of the contact condition; and
responsive to detection of the contact condition, the plunger rod grip assembly is rotated to rotate the plunger rod to connect the plunger rod to the plunger seal.

14. The apparatus of claim 13, wherein, the controller is configured such that, responsive to detection of the contact condition, axial movement of the plunger rod grip assembly is stopped.

15. The apparatus of claim 13, wherein the prime mover is a rotary linear actuator and the contact condition is detected when a position error of the rotary linear actuator exceeds a set value.

16. The apparatus of claim 15, wherein the controller is configured such that rotation of the plunger rod grip assembly continues until a predetermined rotational threshold is reached, at which point the rotating is stopped, and a secondary tightening torque is applied for a set amount of time.

17. The apparatus of claim 16, wherein the rotary linear actuator is carried by a rotating turret that carries the plunger rod grip assembly and the syringe barrel grip assembly.
